(19) United States
(12) Patent Application Publication
(10) Pub. No.: US 2013/0317467 A1
(43) Pub. Date: Nov. 28, 2013

(54) Active Female Incontinence Pad

(71) Applicant: Brooke Campbell, Austin, TX (US)

(72) Inventor: Brooke Campbell, Austin, TX (US)

(73) Assignee: JUSTGOGIRL, LLC, Austin, TX (US)

(21) Appl. No.: 13/480,867

(22) Filed: May 25, 2012

(51) Int. Cl. A61F 13/58; B31D 1/00; A61F 13/511; A61F 13/56; A61L 15/46

(52) U.S. Cl. 604/359; 604/385.03; 604/385.04; 604/378; 604/385.01; 493/374; 493/331

(57) ABSTRACT

A female incontinence pad is adapted to be positioned in a garment with the portion of the pad that is proximate to the urethra being bigger and more absorbent and the portion of the pad spaced apart from the urethra, thereby allowing the pad to have a smaller profile. The pad comprises a body that includes a front section, a middle section and a back section. The front section is adapted to cover the urethra of a user, and is wider and thicker than the back section so as to be able to absorb more liquid than the back section. The middle section has a width and thickness that tapers from the width and thickness of the front section to the width and thickness of the back section. The pad may include one or more adhesive patches.

### BACKGROUND OF THE INVENTION

1. Field of the Invention

The present invention relates to a female incontinence pad adapted to be positioned in a garment.

2. Description of the Related Art

Urinary incontinence, the once-taboo topic of involuntary urine leakage, has become more well known in recent years. But while many are now aware that incontinence affects a large proportion of older people, few realize that it is also a significant problem for active women of all ages. That is, women of all ages can experience involuntary urine leakage during exercising and athletics (also known as athletic incontinence). These women often think that athletic incontinence is a normal part of aging or childbearing, and that they simply have to learn to live with this problem.

Some women who leak during sports or other activities will wear sanitary pads during exercise. Others wear incontinence pads designed for the elderly. Some stop exercising entirely. As sanitary pads are not designed to absorb urine, they often do not fully address the needs of the active women with athletic incontinence. Incontinence pads designed for the elderly tend to be bulky; therefore, many women will not wear them and will avoid exercising, or they will wear bulky clothing not suitable for the activity they are engaging in. Avoiding exercise can lead to future health issues.

### BRIEF DESCRIPTION OF THE DRAWINGS

FIG. 1 is a top view of a female incontinence pad.

FIG. 2 is a bottom view of the female incontinence pad.

FIG. 3 is a side view of the female incontinence pad.

FIG. 4 is a flow chart describing one embodiment of a process for fabricating the structure of FIGS. 1-3.

### DETAILED DESCRIPTION

A female incontinence pad is proposed that is adapted to be positioned in a garment with the portion of the pad that is proximate to the urethra being bigger and more absorbent than the portion of the pad spaced apart from the urethra, thereby allowing the incontinence pad to have a lower profile when worn with athletic apparel. By having a lower profile, the incontinence pad can be worn inside athletic (or other tight fitting) clothing without being visually detectable by others. This technology allows a user to control their athletic incontinence without curtailing athletics or limiting the clothes that can be worn as other people will not know that the user is wearing an incontinence pad.

One embodiment of the pad comprises a body that includes a front section, a middle section and a back section. The front section is adapted to cover the urethra of a user, and is wider and thicker than the back section so as to be able to absorb more liquid than the back section. The middle section has a width and thickness that tapers from the width and thickness of the front section to the width and thickness of the back section. The pad may include one or more adhesive patches.

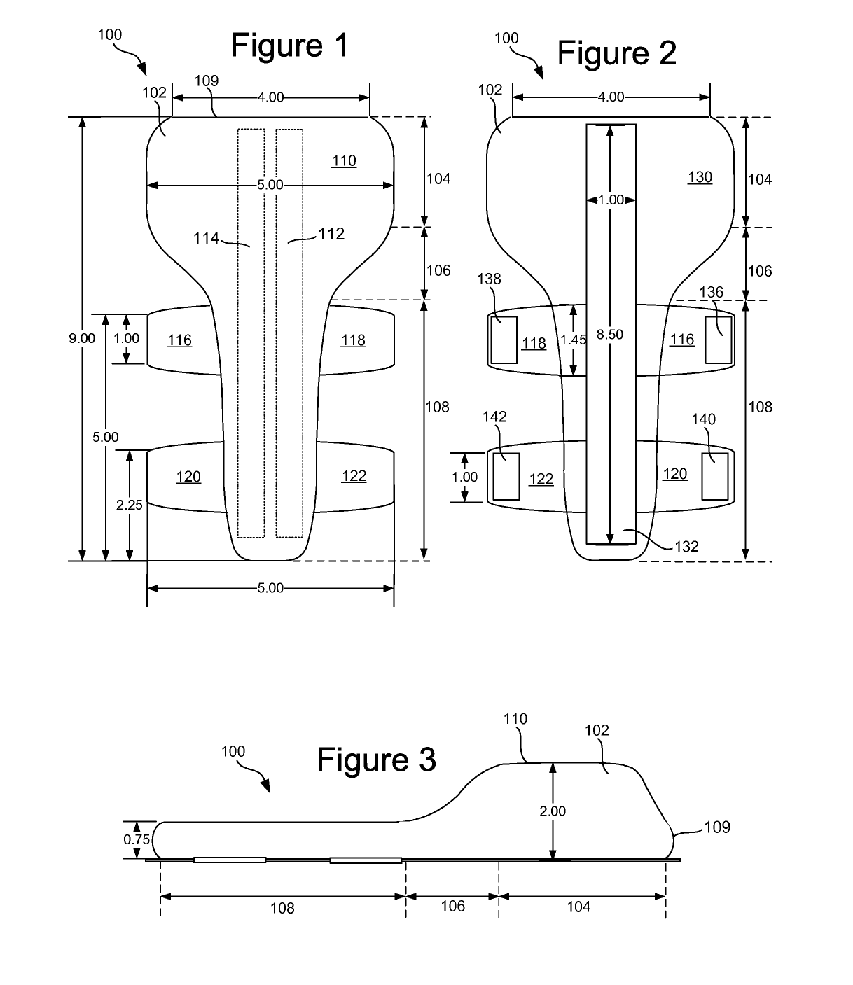

FIG. 1 depicts a top view and FIG. 2 depicts a bottom view of one embodiment of a female incontinence pad 100. Female incontinence pad 100 includes a body 102 having a top surface 110 and a bottom surface 130. In one embodiment, body 102 includes an inner layer of non-woven moisture permeable material, an outer layer of moisture impermeable material, and a layer of moisture absorbent core material. The inner layer and the outer layer are joined (e.g., using an adhesive) along the periphery of body 102 to enclose the layer of moisture absorbent core material. In one example, the layer of moisture absorbent core material comprises cellulose. In some embodiments, the layer of moisture absorbent core material comprises a “super absorbent” polymer and a base or carrier of paper or the like. A non-exhaustive set of examples of non-woven, moisture permeable materials include polyester, polypropylene, rayon fibers, or the like which permit the passage of fluid there through. The outer layer of moisture impermeable material can include moisture impermeable plastic material such as polyethylene, polypropylene or the like. Other materials can also be used and the incontinence pad 100 described herein is not limited to any specific set of materials.

In some embodiments, body 102 can comprise one material throughout. For example, the entire body 102 (or a majority of body 102) can be made of one or more cellulose materials.

In one embodiment, the body may also include a layer of wicking material on the inner surface of the body (proximate to, replacing or comprising) the inner layer of non-woven moisture permeable), to be in contact with the skin of the person wearing the pad 100. In one embodiment, surface 110 can be the wicking material and/or inner layer of non/woven moisture permeable material discussed above.

In one embodiment, body 102 includes a front section 104, a middle section 106, and a back section 108. As depicted in FIGS. 1 and 2, front section 104 is wider than back section 108. It can also be seen that front section 104 and back section 108 are not rectangular and, in some embodiments, can have width that varies within the section. FIG. 1 shows the width of front section 104 to be four (4) inches wide at front surface 109 and five (5) inches wide at its widest point. Thus, the average width of front section 104 is between four and five inches, but closer to five inches. The average width of back section 108 is approximately two inches. Therefore, the average width of front section 104 is larger than the average width of back section 108. Middle section 106 is connected to and between front section 104 and back section 108. The width of middle section 106 is tapered so that it decreases from a width of front section 104 to a width of back section 108 FIG. 1 also shows the length of the entire body to be approximately 9 inches.

FIG. 1 shows two rectangles 112 and 114 in dotted lines. These rectangles represent odor absorbing material that is positioned inside body 102. As can be seen, the odor absorbing material is positioned in front section 104, middle section 106, and back section 108. In some embodiments, body 102 will only include one strip of odor-absorbing material. In other embodiments, there can be multiple strips (or other shapes) of odor-absorbing material. One example of odor-absorbing material includes baking soda. Other types of odor-absorbing material can also be used.

In some embodiments, body 102 will also include a set of wings. In one example, as depicted in FIG. 1, body 102 includes four wings 116, 118, 120, and 122. In one implementation, each of the wings is made of a plastic material, cloth material, or other type of material. FIG. 1 shows the wings to be approximately 1 inch thick and 1½ inches in length. Other dimensions can also be used. In one embodiment, body 102, wing 116, wing 118, wing 120, and wing 122 comprise a single monolithic structure. In other embodiments, each of the wings is a separate structure from body 102 and is attached to body 102 using an adhesive or they are sewn on. In other embodiments, wings 116 and 118 can comprise a single monolithic structure that is attached to body 102, and wings 120 and 122 form a single monolithic structure that is attached to body 102. Wings 116 and 118 are connected to back section 108, and they are proximate to middle section 106. Wings 140 and 142 are also connected to back section 108, but they are spaced apart from middle section 106.

FIG. 2 depicts a bottom view of body 102 of female incontinence pad 100. Body 102 includes bottom surface 130, which is made to contact a garment worn by the user. For example, female incontinence pad 100 is adapted to be positioned in a garment, such as a user's underwear, biking shorts, or other athletic clothing. Bottom surface 130 is intended to be positioned against the surface of the garment while surface 110 is designed to be positioned against the user's body. Along the length of bottom surface 130 (i.e., along the bottom surface of front section 104, middle section 106, and back section 108) is an adhesive patch 132, which is rectangular in shape. In one example, rectangular adhesive patch 132 is 1 inch wide and 8.5 inches long. However, other dimensions and shapes can also be implemented.

Adhesive patch 136 is positioned on the bottom surface of wing 116, adhesive 138 is positioned on the bottom surface of wing 118, adhesive patch 140 is positioned on the bottom surface of wing 120, and adhesive patch 142 is positioned on the bottom surface of wing 122. In one embodiment, the adhesive patches are all initially manufactured with a cover on them such that the user will peel off the cover in order to stick the adhesive patches to the user's garment in order to secure female incontinence pad 100 to the user's garment. In some embodiments, less than five adhesive patches will be used. In some embodiments, there will be adhesive patches 136, 138, 140, and 142 on the wings but no adhesive patch 132 on the bottom surface 130. In other embodiments, pad 100 will include adhesive patch 132 but not adhesive patches 136-142. Other embodiments will include a subset of adhesive patches 136, 138, 140, and 142. In various alternatives, one or more adhesive patches can be in different locations than as depicted in FIG. 2.

In other embodiments, female incontinence pad 100 can be designed to fit in a pouch sewn into the user's garment. In some cases, female incontinence pad 100 can also be loosely placed in the user's underwear such that the shape of female incontinence pad 100 and the shape of the user's body will cause the pad 100 to remain in place.

FIG. 3 is a side view of female incontinence pad 100. FIG. 3 shows that the thickness of front section 104 at its thickest portion is 2 inches, and the thickness of back section 108 is 0.75 inches. The thickness of middle section 106 tapers from the thickness of front section 104 to the thickness of back section 108. In some embodiments, the thickness of any of the sections can vary. However, the average thickness of front section 104 is greater than the average thickness of back section 108.

In some embodiments, back section 108 has a widest width that is less than half the widest width of front section 104, and front section 104 is at least twice as thick as back section 108. In some embodiments, front section 104 is more than twice as thick as back section 108. In some embodiments, all of the portions of body 102 other than front section 104 are thinner (in thickness) and narrower (in width) than front section 104.

In some embodiments, the portions of body 102 other than front section 104 have a smaller profile and can absorb less liquid than front section 104. These other sections have a smaller profile because they are thinner and/or narrower; therefore, there is less material and they are less bulky. These other sections absorb less liquid than front section 104 because there is less material to absorb.

Note that the dimensions depicted in FIGS. 1-3 are one set of example dimensions. Female incontinence pad 100 can be manufactured to other sets of dimensions. Note also that the dimensions depicted in FIGS. 1-3 are all in inches.

In one embodiment, front section 104 is designed to cover the urethra of the wearer in order to receive maximum amount of leaked urine. Back section 108 is designed to be underneath the torso of the wearer and, in some cases, behind the wearer's torso (depending on the size and shape of the wearer). The design of FIGS. 1-3 provides for the thickest and widest portion of pad 100, which provides the most amount of absorption, right in front of the urethra. Those portions of the pad 100 that are away from the urethra (e.g., back portion 108) are much less bulky (narrower and thinner) so that they have a lower profile and will not impact the comfort or look of the user. This design will make it more difficult for people to visually see that the user is wearing pad 100.

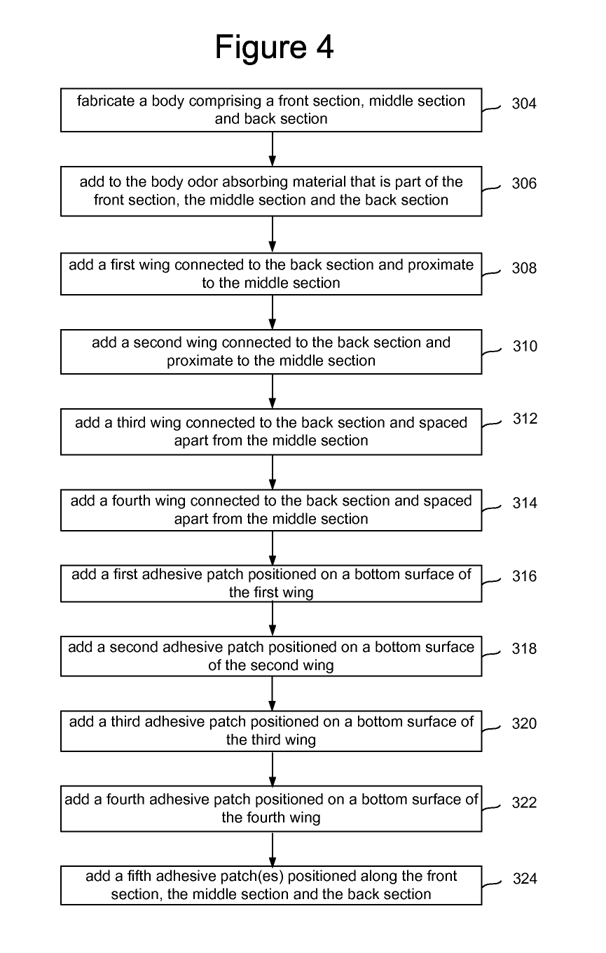

FIG. 4 is a flow chart describing one embodiment of a process for manufacturing female incontinence pad 100. In step 304, body 102 will be fabricated to comprise front section 104, middle section 106, and back section 108. As discussed above, front section 104 will have a first average width and first average thickness, back section 108 will have a second average width and second average thickness, the first average width is greater than the second average width, and the first average thickness is greater than the second average thickness. This arrangement will allow front section 104 to be capable of absorbing more moisture than back section 108. As discussed above, middle section 106 will have a thickness that tapers from the first average thickness to the second average thickness and a width that tapers from the first average width to the second average width.

In step 306, odor-absorbing material is added to body 102. This odor-absorbing material will be added to front section 104, middle section 106, and back section 108. In step 308, a first wing is connected to back section 108 and proximate to middle section 106. In step 310, a second wing is connected to back section 108 and proximate to middle section 106. In step 312, a third wing is connected to back section 108 and spaced apart from middle section 106. In step 314, a fourth wing is connected to back section 108 and spaced apart from middle section 106. In one embodiment, first wing is wing 116, second wing is wing 118, third wing 120, and fourth wing is wing 122. In other embodiments, the wings can be added in different orders and in some cases multiple wings are added at the same time (i.e. when multiple wings are one piece).

In step 316, the first adhesive patch (e.g., adhesive patch 136) is positioned on bottom surface of the first wing (e.g., wing 116). In step 318, a second adhesive patch (e.g., adhesive patch 138) is positioned on the bottom surface of the second wing (e.g. wing 118). In step 320, a third adhesive patch (e.g., adhesive patch 140) is positioned on the bottom surface of the third wing (e.g., wing 120). In step 322, a fourth adhesive patch (e.g., adhesive patch 142) is positioned on the bottom surface of the fourth wing (e.g., wing 122).

In step 324, a fifth adhesive patch is positioned along front section 104, middle section 106, and back section 108. For example, adhesive patch 132 can be positioned on bottom surface 130 of body 102. In some embodiments, multiple adhesive patches can be positioned on bottom surface 130 along front section 104, middle section 106, and back section 108 in step 324.

In some embodiments, female incontinence pad 100 can be manufactured in different colors so that a user can choose a color to match the garment being worn. In some implementations, female incontinence pad 100 can be a disposable device for one-time use. In other embodiments, it can be designed for multiple uses.

One embodiment includes a female incontinence pad adapted to be positioned in a garment. The female incontinence pad comprises a body and a first adhesive patch. The body includes a front section adapted to cover a urethra of a user and a back section. The front section is wider and thicker than the back section so as to be able to absorb more liquid than the back section. The body further comprises a middle section connected to and between the front section and the back section. The middle section has a width that tapers from a width of the front section to a width of the back section. The first adhesive patch is connected to the body and is adapted to adhere the incontinence pad to the garment.

One embodiment includes a female incontinence pad adapted to be positioned in a garment. The female incontinence pad comprises a front section and a back section. The front section has a first average width and a first average thickness. The back section has a second average width and a second average thickness. The first average width is greater than the second average width and the first average thickness is greater than the second average thickness such that the front section is capable to absorbing more moisture than the back section. In one set of implementations, the front section and back section form a body that also includes a middle section having a width and thickness that tapers from the front section to the back section.

One embodiment includes a method of fabricating a female incontinence pad. The method comprises fabricating a body that includes a front section and a back section. The front section has a first average width and a first average thickness. The back section has a second average width and a second average thickness. The first average width is greater than the second average width and the first average thickness is greater than the second average thickness such that the front section is capable to absorbing more moisture than the back section.

The foregoing detailed description has been presented for purposes of illustration and description. It is not intended to be exhaustive or limiting to the precise form disclosed. Many modifications and variations are possible in light of the above teaching. The described embodiments were chosen in order to best explain the principles of the disclosed technology and its practical application to thereby enable others skilled in the art to best utilize the technology in various embodiments and with various modifications as are suited to the particular use contemplated. It is intended that the scope be defined by the claims appended hereto.

What is claimed is:

1. A female incontinence pad adapted to be positioned in a garment, comprising:
a body comprising a front section adapted to cover a urethra of a user and a back section, the front section is wider and thicker than the back section so as to be able to absorb more liquid than the back section, the body further comprising a middle section connected to and between the front section and the back section, the middle section has a width that tapers from a width of the front section to a width of the back section; and
a first adhesive patch connected to the body and adapted to adhere the incontinence pad to the garment.

2. The female incontinence pad of claim 1, wherein:
the front section is at least twice as thick as the back section.

3. The female incontinence pad of claim 1, wherein:
the thickness of the middle section tapers from a thickness of the front section to a thickness of the back section.

4. The female incontinence pad of claim 1, further comprising:
wings connected to the body, the first adhesive patch is positioned on one of the wings; and
a second adhesive patch positioned on a different wing than the first adhesive patch and adapted to adhere the incontinence pad to the garment.

5. The female incontinence pad of claim 4, wherein:
the body and wings form a single unitary component.

6. The female incontinence pad of claim 4, wherein:
the body and wings are separate structures that are connected together.

7. The female incontinence pad of claim 4, wherein:
the wings include a first wing connected to the back section and proximate to the middle section, a second wing connected to the back section and proximate to the middle section, a third wing connected to the back section and spaced apart from the middle section and a fourth wings connected to the back section and spaced apart from the middle section
the first adhesive patch is positioned on a bottom surface of the first wing;
the second adhesive patch is positioned on a bottom surface of the second wing; and
the female incontinence pad further comprises a third adhesive patch positioned on a bottom surface of the third wing and a fourth adhesive patch positioned on a bottom surface of the fourth wing.

8. The female incontinence pad of claim 7, further comprising:
a fifth adhesive patch positioned on a bottom surface the front section, the middle section and the back section.

9. The female incontinence pad of claim 8, wherein:
the body further comprises an odor absorbing material positioned in the front section, the middle section and the back section.

10. The female incontinence pad of claim 1, wherein:
the first adhesive patch is positioned on a bottom surface of the front section, the middle section and the back section.

11. The female incontinence pad of claim 1, wherein:
the body further comprises odor absorbing material that is part of the front section, the middle section and the back section.

12. The female incontinence pad of claim 1, wherein:
the portions of the body other than the front section are narrower and less absorbent of liquid than the front section.

13. The female incontinence pad of claim 1, wherein:
the portions of the body other than the front section have a smaller profile and are less absorbent of liquid that the front section.

14. The female incontinence pad of claim 1, wherein:
the back section has a widest width that less than half of a widest width of the front section; and
the front section is at least twice as thick as the back section.

15. The female incontinence pad of claim 1, wherein:
the body comprises an inner layer of non-woven moisture permeable material, an outer layer of moisture impermeable material and a layer of moisture absorbent material positioned between the inner layer and outer layer; and
the body further comprising a layer of moisture wicking material proximate to the inner layer and forming an inner surface of the body.

16. A female incontinence pad adapted to be positioned in a garment, comprising:
a front section having a first average width and a first average thickness; and
a back section having a second average width and a second average thickness, the first average width is greater than the second average width and the first average thickness is greater than the second average thickness such that the front section is capable to absorbing more moisture than the back section.

17. The female incontinence pad of claim 16, further comprising:
a middle section having a width the tapers from the front section to the back section;
a first wing connected to the back section and proximate to the middle section;
a second wing connected to the back section and proximate to the middle section;
a third wing connected to the back section and spaced apart from the middle section;
a fourth wing connected to the back section and spaced apart from the middle section;
a first adhesive patch positioned on a bottom surface of the first wing;
a second adhesive patch positioned on a bottom surface of the second wing;
a third adhesive patch positioned on a bottom surface of the third wing;
a fourth adhesive patch positioned on a bottom surface of the fourth wing;
a fifth adhesive patch positioned along the front section, the middle section and the back section; and
odor absorbing material positioned in the front section, the middle section and the back section.

18. A method of fabricating a female incontinence pad, comprising:
fabricating a body comprising a front section and a back section, the front section having a first average width and a first average thickness, the back section having a second average width and a second average thickness, the first average width is greater than the second average width and the first average thickness is greater than the second average thickness such that the front section is capable to absorbing more moisture than the back section.

19. The method of claim 18, further comprising:
adding a first adhesive to an outer surface of the body to adhere the body to a garment.

20. The method of claim 18, wherein:
the fabricating the body further comprises creating a middle section connected to and positioned between the front section and the back section with a width that tapers from the first average width to the second width and a thickness that tapers from the first average thickness to the second average thickness.

21. The method of claim 20, further comprising:
adding odor absorbing material to the body, the odor absorbing material is positioned in the front section, the middle section and the back section.

22. The method of claim 20, further comprising:
adding a first wing connected to the back section and proximate to the middle section;
adding a second wing connected to the back section and proximate to the middle section;
adding a third wing connected to the back section and spaced apart from the middle section;
adding a fourth wing connected to the back section and spaced apart from the middle section;
adding a first adhesive patch to a bottom surface of the first wing;
adding a second adhesive patch to a bottom surface of the second wing;
adding a third adhesive patch to a bottom surface of the third wing;
adding a fourth adhesive patch to a bottom surface of the fourth wing; and
adding a fifth adhesive patch to the front section, the middle section and the back section.
